(19) United States
(12) Patent Application Publication
(10) Pub. No.: US 2014/0379072 A1
(43) Pub. Date: Dec. 25, 2014

(54) Tissue-Engineered Vascular Graft and Its Fabrication Approach

(71) Applicant: FGBU "NII Kompleksnykh Problem Serdechno- Sosudistykh Zabolevany Sibirskogo Otdeleniya RAMN", Kemerovo (RU)

(72) Inventors: Leonid S. BARBARASH, Kemerovo (RU); Jacob L. ELGUDIN, Solon, OH (US); Victoria V. SEVOSTYANOVA, Novokuznetsk (RU); Alexey S. GOLOVKIN, Kemerovo (RU)

(21) Appl. No.: 14/480,329

(22) Filed: Sep. 8, 2014

Related U.S. Application Data
(63) Continuation of application No. PCT/RU2013/000250, filed on Mar. 27, 2013.

(30) Foreign Application Priority Data
Apr. 6, 2012 (RU) 2012113439

(51) Int. Cl. A61F 2/06; A61L 31/06; D01D 5/00

(52) U.S. Cl. CPC A61F 2/06; D01D 5/0007; A61L 31/06; D10B 2331/041; D10B 2401/12
USPC 623/1.38; 264/465

(57) ABSTRACT

Tissue-engineered vascular graft is designed to be used in cardiovascular surgeries, especially in coronary artery bypass grafting and peripheral vessels reconstruction procedures. Two-phase electrospinning technique was employed to fabricate a biodegradable polymer graft composed of the porous tubular scaffold supplemented by biologically active molecules, incorporated directly into the matrix walls in order to promote regeneration process of the patient's own vessel wall.

### RELATED APPLICATIONS

This Application is a Continuation application of International Application PCT/RU2013/000250, filed on Mar. 27, 2013, which in turn claims priority to Russian Patent Applications No. RU 2012113439, filed Apr. 6, 2012, both of which are incorporated herein by reference in their entirety.

### FIELD OF THE INVENTION

Field of the invention: medicine and tissue engineering, may be used in cardiovascular surgeries, especially in coronary artery bypass grafting and peripheral artery reconstructions.

### BACKGROUND OF THE INVENTION

Nowadays, autologous veins and arteries as well as vessels of xenogenic origin are generally used for the surgical treatment of cardiovascular diseases associated with atherosclerotic occlusion of the peripheral and coronary arteries. An average lifespan of a tissue prosthesis is 5 years preconditioning the need of a repeat revascularization (Bokeria L. A., A high percentage of re-operation in patients with coronary artery disease—current problems/Bokeria L. A., and Berishvili I. I., Solnychkov et al.//Bulletin of Bakoulev CCVS for Cardiovascular Surgery.-2009.-No. 10 (3.)-P.5-27).

The use of synthetic materials for vascular prostheses, such as polytetrafluoroethylene (PTFE) or Dacron, may overcome this problem, however, small-diameter vascular grafts (less than 6 mm) are susceptible to intraluminal clotting.

Currently, tissue-engineered vascular grafts can become a viable alternative to autologous and xenogenic veins and arteries as well as synthetic vascular prostheses in cardiac surgeries. The principle goal of tissue engineering is to produce a completely cell-derived vascular graft that may be used in cardiovascular surgeries. The set goal resulted in attempts to elaborate an absorbable graft seeded with a patient's own cells.

The tissue-engineered vascular graft was reported to have been fabricated from a biocompatible, biodegradable scaffold seeded with auto-cells of one or several types, derived from bone marrow or peripheral blood of a patient (U.S. Application 20090275129A1, IPC C12N5/08, C 12N5/06, publ. May 11, 2009). The biocompatible scaffold was composed of natural or synthetic biodegradable polymers with porous structure. The cells derived from the patient for further seeding onto the scaffold were cultured under sterile conditions till their expansion, and then were seeded onto the surface.

The scaffold was placed in a bioreactor to archive cell proliferation and extracellular matrix formation. The complication to collect sufficient cellular material from the patient, prolonged cell culture and seeding time onto the scaffold marked a major disadvantage to construct a complex blood vessel substitute.

The closest invention to the claimed technical decision appears to be a bioengineered vascular graft designed for the implantation into the patient's organism, serving as a basis for further regeneration with capability to grow into a functional vessel (U.S. Application 20060100717A1, IPC A61F2/06, publ. Nov. 5, 2006).

The tubular scaffold of bioengineered graft was manufactured from collagen and partially seeded with the donor cells. Since the graft had been implanted, it underwent polymer biodegradation occurring concomitantly with remodeling of the damaged vessel segment by depositing the collagen matrix with the host's cells. The collagen scaffold would degrade at the same rate, as the natural tissue would proliferate.

The major disadvantage of that vascular graft design is the utilization of natural polymeric scaffold, fabricated from collagen, which, in its turn, does not have sufficient flexibility and strength limiting the graft's application in cardiovascular surgery. Moreover, the production of tissue-engineered animal collagen-based grafts results in immune response and allergic reactions to the implanted graft. In addition, the partial cells seeding of the collagen scaffold gives rise to several issues: invasive and painful procedures for collecting biological material from a patient or a donor, the risk of infection.

### SUMMARY OF THE INVENTION

The technical result of the invention relates to a fabrication of tissue-engineered small-diameter vascular graft of high patency and long lifespan for bioremodeling of the damaged blood vessel segment in vivo. The set goal is archived by employing two-phase electrospinning technique to fabricate the porous tubular scaffold from biodegradable polymer supplemented by biologically active molecules, incorporated directly into the matrix wall to promote regeneration process of the vessel wall.

### BRIEF DESCRIPTION OF THE DRAWINGS

The description particularly refers to the accompanying figures in which

FIG. 1 represents a general view of the vascular graft as a hollow tube the vascular graft scaffold,

FIG. 2—the porous graft wall, composed of the biopolymer fibers spun by the electrospinning,

FIG. 3—biomolecules incorporated into the polymer fiber.

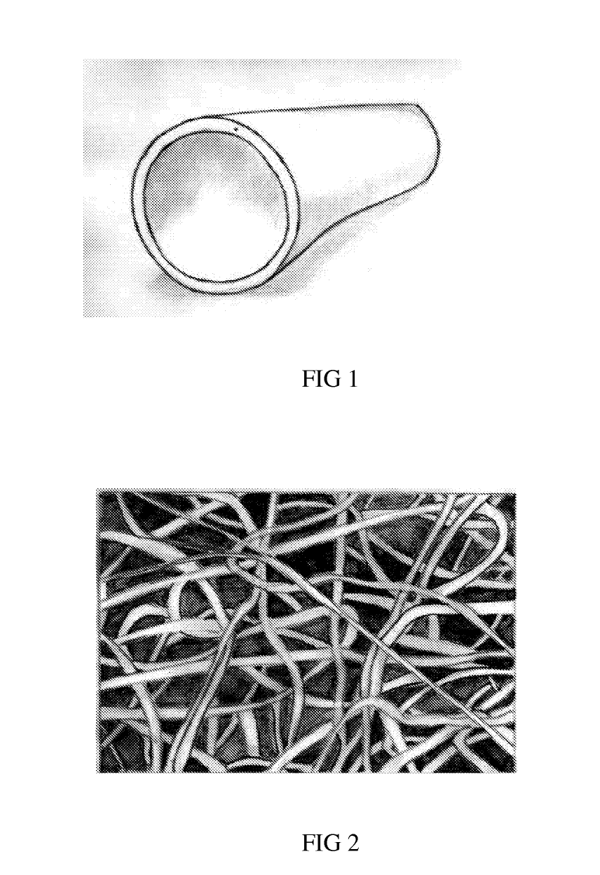

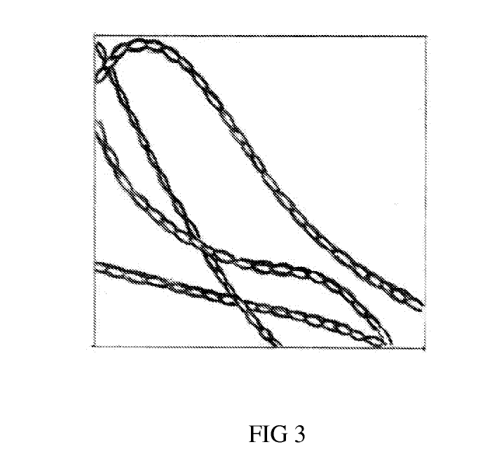

### DETAILED DESCRIPTION OF THE PREFERRED EMBODIMENTS

A synthetic polymer with a long biodegradation period-polycaprolactone (poly(e-caprolactone (PCL) is used to fabricate vascular graft scaffolds due to its suitable and well-known mechanical properties (flexibility and strength). Moreover, this polymer is biocompatible and bioresistant, the degradation rate of PCL fibers, spun by electrospinning, is from three months up to a year. This PCL degradation rate contributes to the long-term support of the necessary mechanical graft properties to grow into functional native vessel; the polymer's hydrolytic degradation and blood vessel regeneration are concomitant and coordinated in time. No toxic substances are formed as a result of the biodegradation: water and caproic acid.

The electrospining technique used to fabricate scaffolds enables to spin micro- and nanofibers and porous structures from polymer solutions and melts. This method relates to fiber fabrication within a strong electric field generated between two electrodes bearing electrical charges of opposite polarity, with one electrode being placed into the spinning polymer solution or melt, the other being attached to a metal collector. Vascular graft scaffolds are produced on the electrospinning apparatus, a polymer solution is loaded in a syringe, a metering pump attached to a plunger generates pressure at a given rate.

An electrical potential is applied to a blunt-end needle attached to the syringe. The polymer is ejected from the tip, solidifies and leaves a fiber behind. The polymer fibers are captured on a rotating collector screen with the other attached electrode to comprise a porous material. The pores size do not exceed 20 μm in order to prevent bleeding through the prosthesis wall.

The following parameters of electrospinning are used to produce vascular grafts: voltage—10-50 kV, the flow rate of the polymer solution—1-10 ml/h, the distance between the capillary and collection screen—1-20 cm, motion of the target screen—about 10-300/min

Polymer fibers are supplemented with biological molecules such as vascular endothelial growth factor (VEGF), fibroblast growth factor beta (b-FGF), stromal derived factor-1alpha (SDF-1α) as well as heparin molecules during the electrospinning process. VEGF incorporation into the scaffold structure contributes to its faster endothelialization, because this growth factor has been shown to play an important role in the regulation of endothelial cells migration and proliferation. In addition, bFGF is used for induction of endothelial cells and fibroblasts proliferation. SDF-1αactivates autologous stem cells migration towards area of an injured segment, promoting its healing.

The incorporation of heparin molecules into the matrix wall reduces the risk of the graft lumen thrombosis. The incorporation of these growth factors and heparin into the graft wall is performed by mixing the biodegradable polymer solution with the solution containing biological molecules in phosphate-buffered saline at a ratio of 20:1, then the electrospinning is applied. Since each type of biomolecules has its own impact on host's cells, the proposed vascular graft may be composed of either one type of molecules or their combination.

During the polymer biodegradation process the incorporated molecules are released into the surrounding tissues and perform their biological functions, such as stimulation and regulation of the native vessel growth. Furthermore, the molecules attached to the polymer fibers have no contact with the external environment that ensures the long-term preservation of their functions promoting to perform grafts sterilization prior to the implantation.

The utilization of polycaprolactone for a graft fabrication eliminates the immune response and allergic reactions caused by the implantation. Continuous delivery of bioactive molecules into the surrounding tissues is ensured by the low rate of polymer biodegradation.

The trial of the PCL vascular grafts was conducted in the Louis Stokes Cleveland Veterans Affairs Medical Center, Cleveland, Ohio, USA.

Example 1. Vascular grafts (internal diameter of 2 μm, thickness 100 μm) composed of biodegradable polycaprolactone polymer (PCL) (M=80.000) were fabricated by electrospinning method and implanted in five male inbred Wistar rats (weight 400-450 g). PCL-grafts were implanted in the abdominal aorta between the renal artery and the aortic bifurcation. After the clamps were removed, the blood flow through the grafts was assessed by Doppler ultrasonography.

After 6 weeks, the animals were euthanased, the anastomoses and graft performance were assessed by the histological examination of the samples stained with haematoxylin and eosin, Mallory and Van Gieson technique.

The histological examination of the graft lumen and anastomoses areas revealed the continuous layer of neointima. The inner surface of the seeded graft was covered with endothelial cells, most of which had enlarged hyperchromatic nuclei and decreased nuclear-cytoplasmic index compared to the endothelial cells of the own aorta.

The graft was infiltrated by the cells of myofibroblasts and macrophages morphological features. Collagen accumulation sites, rich in glycosaminoglycans, laminin and fibronectin, were detected on the whole graft surface. The macroscopic evaluation of the implanted graft in the perivascular tissue found no signs of bleeding.

Thus, the conducted trial reported the structures formation on the PCL grafts that mimic native blood vessels; hence, these polymer substitutes appears to be promising for use in cardiovascular surgeries as a tissue-engineered vascular grafts.

What is claimed is:

1. A method of producing a tissue-engineered vascular graft, the method comprising:
applying an electrospining process to fabricate biodegradable polymer scaffold structures having porous walls;
incorporating in the scaffold structures biological molecules of at least one of: vascular endothelial growth factor (VEGF), fibroblast growth factor beta (b-FGF) and stromal derived factor-1alpha (SDF-1α), or heparin molecules; and
forming the graft using the scaffold structures containing the incorporated molecules.

2. The method of claim 1, wherein the biodegradable polymer comprises polycaprolactone (PCL).

3. The method of claim 1, wherein the scaffold structures comprise fibers or micro-fibers.

4. The method of claim 1, further comprising:
incorporating the molecules during the electrospinning process of fabricating the scaffold structures.

5. The method of claim 4, further comprising:
mixing a solution of the biodegradable polymer with a solution containing the VEGF, b-FGF, SDF-1α or heparin molecules in phosphate-buffered saline at a ratio of about 20:1.

6. The method of claim 4, further comprising:
using the electrospining process providing: voltages in a range of 10-50 kV, flow rates in a range of 1-10 ml/h, and distances between capillary and collection screens in a range of 1-20 cm.

7. The method of claim 1, further comprising:
forming the graft having an internal diameter in a range of 2-6 μm.

8. A tissue-engineered vascular graft comprising biodegradable polymer scaffold structures (i) having porous walls and (ii) incorporating biological molecules of at least one of: vascular endothelial growth factor (VEGF), fibroblast growth factor beta (b-FGF) and stromal derived factor-1alpha (SDF-1α), or heparin molecules.

9. The graft of claim 8, wherein the graft has a tubular form factor.

10. The graft of claim 8, wherein the scaffold structures comprise fibers or micro-fibers.

11. The graft of claim 8, wherein the biodegradable polymer comprises polycaprolactone (PCL).
